(19) United States
(12) Patent Application Publication
(10) Pub. No.: US 2011/0293691 A1
(43) Pub. Date: Dec. 1, 2011

(54) MULTIMODAL ADHESION BARRIER

(76) Inventors: Kurt R. Weber, Winston-Salem, NC (US); Steve J. Hodges, Winston-Salem, NC (US); Scott A. Washburn, Pfafftown, NC (US)

(21) Appl. No.: 13/096,301

(22) Filed: Apr. 28, 2011

Related U.S. Application Data
(60) Provisional application No. 61/328,840, filed on Apr. 28, 2010.

(51) Int. Cl. A61K 9/00; A61K 31/407; A61K 31/196; A61K 31/517; B65D 81/20; A61K 31/275; A61K 31/513; A61K 31/704; A61P 35/00; B65D 85/00; A61K 9/70; A61K 31/198

(52) U.S. Cl. 424/443; 424/400; 514/410; 514/563; 514/266.22; 514/562; 514/526; 514/274; 514/33; 206/524.6; 206/524.8

(57) ABSTRACT

Provided herein are multimodal barrier materials useful for preventing adhesions in a subject, which may include a collagen, a collagen deposition inhibitor and/or a chitosan.

### RELATED APPLICATIONS

This patent application claims the benefit under 35 U.S.C. §119(e) of U.S. Provisional Application No. 61/328,840, filed Apr. 28, 2010, the disclosure of which is hereby incorporated by reference in its entirety.

### FIELD OF THE INVENTION

The present invention concerns the prevention, inhibition and/or treatment of adhesions.

### BACKGROUND

The problem of intra-abdominal adhesion formation following surgery has been known for centuries, and the first published reports describing the use of various agents as adjuvants for adhesion prevention began to appear in the latter half of the 19th century (Becker and Stucci, “Intra-abdominal Adhesion Prevention: Are We Getting Any Closer?” Annals of Surgery, 240(2):202-204). Despite this long history, few treatments have proven effective against adhesions. This is a reflection of how little is known about the mechanisms of adhesion formation.

Currently, surgeons endeavor to prevent adhesions with the use of improved surgical technique, along with the use of a protective physical barrier. Recent anti-adhesion barrier products include Seprafilm™ barrier (Genzyme Corporation), Interceed™ barrier (Johnson & Johnson), NeuraWrap™ nerve protector (Integra LifeSciences Corp.), and TenoGlide™ tendon protector (Integra LifeSciences Corp.). Seprafilm™ barrier is a clear sheet made of hyaluronic acid and carboxymethylcellulose. It forms a physical barrier and is absorbed by the body in under 30 days. Interceed™ barrier is a bioabsorbable sheet made from a rayon material (oxidized regenerated cellulose). NeuraWrap™ nerve protector is a bioabsorbable collagen wrap, and TenoGlide™ tendon protector sheet is a matrix of crosslinked collagen and glycosaminoglycan. Anti-adhesion barrier gels include Oxiplex® (FzioMed, Inc.), which is made of carboxymethylcellulose and polyethylene oxide, and Adcon-L (Gliatech), which is a polyglycan ester and absorbable pig-derived gelatin in phosphate-buffered saline.

Despite the long history of adhesions, the numerous attempts to combat them, and the available anti-adhesion products on the market, they remain a serious and common complication of surgery. For example, many studies have reported that up to 94% of patients develop primary abdominal adhesions following laparotomy. Clearly, better options to combat the recurring problem of adhesions are needed.

### SUMMARY

Provided herein are improved barrier materials for preventing or inhibiting adhesions in a subject. In some embodiments, the barrier material is mulitmodal. In some embodiments, the material includes one or more of collagen (e.g., amnion), a collagen deposition inhibitor (e.g., mithramycin, mitomycin-c, tranilast, halofuginone, d-penicillamine, beta-aminopropionitrile, okadaic acid, LY294002 (PI-3K inhibitor), 5-fluorouracil, or analogs thereof) and a chitin or derivative thereof such as chitosan. In some embodiments, the material is bioabsorbable. In some embodiments, the material is in the form of a sheet, a sheath or a plug.

Also provided are methods of preventing or inhibiting adhesions (e.g. pelvic, abdominal, peritendinous, perineuroal, etc.) in a subject in need thereof comprising administering a barrier material described herein to said subject in an amount effective to prevent said adhesions. In some embodiments, administering includes contacting said barrier material to a tissue or positioning said adhesion barrier between tissues during surgery (e.g., abdominal surgery, pelvic or gynecological surgery, orthopedic surgery, ocular surgery, neurosurgery, urologic surgery, cardiothoracic surgery, plastic surgery, veterinary surgery, otolaryngology surgery, podiatric surgery, vascular surgery, trauma surgery, transplant surgery, etc.). In some embodiments, the barrier material is preformed.

Further provided are kits which include a barrier material as described herein packaged in a container. In some embodiments, the material is packaged in sterile form. In some embodiments, the material is provided in dehydrated form, which may optionally be hydrated prior to use. In some embodiments, the container is vacuum-packed. In some embodiments, the container includes a plastic or foil. In some embodiments, the material is provided which includes a single unit dose of a collagen deposition inhibitor.

Also provided is the use of a barrier material as described herein for the treatment or prevention of adhesions in a subject in need thereof.

### BRIEF DESCRIPTION OF THE DRAWINGS

FIG. 1 presents exemplary embodiments of the adhesion barrier. 1A gives examples of sizes of different adhesion barrier sheets. 1B provides examples of multimodal layer configurations. 1C illustrates a putty/powder embodiment.

FIGS. 2A-2C illustrates an example of an open tube adhesion barrier formation formed in a mold (5) having a flat or substantially flat portion (10) adjacent to one or more sides of a concave portion (20); and in use as the adhesion barrier is closed around a nerve, tendon, blood vessel, etc. (2B-2C).

FIG. 2D illustrates another exemplary mold configuration with a flat or substantially flat portion (10) adjacent to one or more sides of a concave portion (20), which may be used to form a rounded adhesion barrier for ease of application to a rounded tissue such as an organ.

FIG. 3 presents exemplary embodiments of an open tube adhesion barrier embodiment having various configurations of the layers. “A” represents a collagen component, “B” represents a collagen deposition inhibitor component, and “C” represents a chitosan component. A mixed gel indicated as “B”+“C” includes both a collagen deposition inhibitor and chitosan component. The barrier may optionally be molded to form an open tube with an overall length of x and a diameter y of the semicircular portion.

FIG. 4 illustrates embodiments of the adhesion barrier in which a powder chitosan component or layer (“C(pw)”) or putty chitosan component or layer (“C(pt)”) is included.

FIG. 5 provides exemplary products in the form of putty rectangular prism or cylinder that can optionally be shaped and are useful to fill and form to defects or crevices as needed. Other polyhedron shapes may be provided, as well (e.g., other pentahedrons such as a triangular prism).

### DETAILED DESCRIPTION OF THE PREFERRED EMBODIMENTS

Provided herein are improved adhesion barriers. In some embodiments, a multimodal approach is used for adhesion prevention and/or treatment, with two or more layers and/or components being applied or included in the barrier.

For example, an embodiment of an adhesion barrier provided herein has one, two, three, or four or more layers, which layers may be the same or different and provided in any arrangement. Exemplary embodiments, with “A” referring to the collagen component described below in section A, “B” referring to the collagen deposition inhibitor component described below in section B, and “C” referring to the chitosan component described below in section C, include the following: AB; BAB; ABA; BAC; BAAC; etc.

In some embodiments, the collagen deposition inhibitor and chitosan components may be provided together in a mixed gel, e.g., A(B+C) or (B+C)A(B+C).

As disclosed herein, it has been unexpectedly found that some embodiments of the multimodal combinations of the components taught herein have significant and/or synergistic anti-adhesion properties as compared to their use as individual components.

In some embodiments, the barrier material may be a sheet or sheath material or plug that includes, e.g., a layer of amnion in between two layers of chitosan, i.e., chitosan/amnion/chitosan or CAC. In some embodiments, the material may include a layer of amnion in between two layers of halofuginone, i.e., halofuginone/amion/halofuginone or HAH. In some embodiments, the material may include a layer of amion between one layer of chitosan and one layer of halofuginone, i.e., chitosan/amnion/halofuginone sheets (CAH).

The disclosures of all United States Patent references cited herein are hereby incorporated by reference herein as if fully set forth. As used herein in the description of the invention and the appended claims, the singular forms “a,” “an” and “the” are intended to include the plural forms as well, unless the context clearly indicates otherwise. Furthermore, the terms “about” and “approximately” as used herein when referring to a measurable value such as an amount of a compound, dose, time, temperature, and the like, is meant to encompass variations of 20%, 10%, 5%, 1%, 0.5%, or even 0.1% of the specified amount. Also, as used herein, “and/or” refers to and encompasses any and all possible combinations of one or more of the associated listed items, as well as the lack of combinations when interpreted in the alternative (“or”).

“Adhesion barrier” or “barrier” refers to materials useful for the prevention or treatment of adhesions, which include physical barrier materials, gels, etc.

“Adhesions” are fibrous bands that form between tissues and organs. Often the result of injury sustained by the tissue during surgery, adhesions may also form due to radiation, infection, inflammation, trauma or disease (e.g., pelvic inflammatory disease may cause abdominal adhesions).

Abdominal adhesions are associated with chronic abdominal and pelvic pain, infertility, and adhesive small bowel obstruction (ASBO).

“Subjects” that may be treated by the present invention include both human subjects for medical purposes and animal subjects for veterinary and laboratory purposes. Other suitable animal subjects are, in general, mammalian subjects such as primates, bovines, ovines, caprines, porcines, equines, felines, canines, lagomorphs, rodents (e.g., rats and mice), etc. Human subjects include fetal, neonatal, infant, juvenile, adult and geriatric subjects.

“Preventing”, “inhibiting” or “treating” adhesions refers to any type of treatment that imparts a benefit to a subject afflicted with or at risk of developing adhesions or complications involving scar tissue production and/or collagen production associated with the development of adhesions, including improvement in the condition of the subject (e.g., in one or more symptoms), delay or inhibition of the progression of adhesion development, delay in the onset or the amelioration of symptoms or slowing in the progression of symptoms, etc. As used herein, “treatment” and “prevention” are not necessarily meant to imply cure or complete abolition of symptoms, but refer to any type of treatment that imparts a benefit to a patient afflicted with adhesions or complications associated therewith, including improvement in the condition of the patient (e.g., in one or more symptoms), delay in the progression of adhesion formation, etc.

“Treatment effective amount”, “prevention effective amount”, “amount effective to treat”, “amount effective to prevent”, “amount effective to inhibit” or the like as used herein means an amount of the material or composition sufficient to produce a desirable effect upon a patient inflicted with or at risk for developing adhesions.

In some embodiments, the adhesion barrier is contacted to a tissue, or positioned between tissues, during surgery. Embodiments of the adhesions barrier provided herein are useful in a variety of surgeries, e.g., in general (abdominal) surgery, pelvic or gynecological surgery, orthopedic surgery, ocular surgery, or neurosurgery, urologic surgery, cardiothoracic surgery, plastic surgery, veterinary surgery, otolaryngology surgery, podiatric surgery, vascular surgery, trauma surgery, transplant surgery, etc., for application to injured and/or exposed tissue.

“Pharmaceutically acceptable” as used herein means that the material or composition is suitable for administration to a subject to achieve the treatments described herein, without unduly deleterious side effects in light of the severity of the disease and necessity of the treatment.

Materials used to form the adhesion barrier may be preformed or formed in situ. See, e.g., U.S. Pat. No. 6,638,917. Materials may be biodegradable and/or bioabsorbable (e.g., a hemostatic material) or non-bioabsorbable (e.g., a non-absorbable mesh, such as is currently used in hernia repair). A biodegradable material is capable of being broken down into smaller or elemental components by a host. In some embodiments, the materials are bioabsorbable (absorbed by the body) within a time of from 1, 2, 3, 4 or 5 days to 7, 10, 14, 21, 25 or 28 days. In some embodiments, the materials are bioabsorbable within a time of from 8, 10 or 12 days to 18, 20 or 22 days.

### I. Components

### A. Collagen

The “collagen” component as used herein refers to a material in which a substantial portion (e.g., 30, 50, 70, 80, or 90% or more by weight) of the structural matrix is collagen. Collagen may be synthetic or naturally derived, e.g., from a natural tissue (with or without decellularization). Collagen (e.g., Type I, Type II, Type III, etc.) is the major protein component of the extracellular matrix in organisms.

Collagen derived from natural tissue may be autologous or autogeneic (i.e., from the subject to be treated), isogeneic (i.e., a genetically identical but different subject, e.g., from an identical twin), allogeneic (i.e., from a non-genetically identical member of the same species) or xenogeneic (i.e., from a member of a different species) with respect to the subject being treated therewith. “Natural tissues” are tissues that are normally found in an animal without human manipulation. Tissues that may be used may be from any suitable animal source, including human, other mammalian (e.g., cat, dog, pig, cow, sheep, horse, monkey), avian (e.g., chicken, turkey, duck, goose, etc.), reptile, amphibian, etc.

In some embodiments, the collagen material is, or is derived from, all or a portion of the placental membrane, particularly the amnion portion of the placental membrane. The placental membrane is a thin, tough, transparent membrane, typically 20-500 micrometers thick in humans, and is composed of the amnion and the chorion. The amnion, the innermost layer of the placental membrane, is typically 10-80 micrometers thick in humans.

Amnion has low or no antigenicity, and has been used medically for decades, for example, as a skin substitute, in the treatment of burns, and for the repair of conjuctival defects. Amnion has proven effective in preventing scleral scarring following pterygium removal (Tseng, Amniotic membrane transplant for ocular surface reconstruction. Biosci Rep 2001; 21:481-9). Human amnion has also been shown to prevent adhesions in animal models of abdominal incisional hernia repair using polypropylene mesh (Szabo et al., Evaluation of Seprafilm and Amniotic Membrane as Adhesion Prophylaxis in Mesh Repair of Abdominal Wall Hernia in Rats. Eur Surg Res 2000; 32:125-8).

A variety of preparations and storage conditions for amnion are known. These include the use of freshly-harvested membrane, cold-stored (e.g., with rinsing in a 0.025% solution of sodium hypochlorite and stored at 4° C. in sterile solution with antibiotic(s)), dried in open air, frozen (e.g., flash frozen in liquid nitrogen), freeze dried (lyophilized) (e.g., at −60° C. under vacuum for 48 hours) then irradiated (e.g., 2.5 mega rads (25 K Gray) in a batch-type cobalt-60 irradiator), and stabilized or crosslinked amion (e.g., with glutaraldehyde treatment). Amion may also be low-heat dehydrated, which resulting dehydrated membrane can be stored at room temperature for prolonged periods of time prior to use (see John et al. Ultrastructural findings of new “free-standing,” low-heat dehydrated human amniotic membrane. ARVO Abstracts 2002; John T. Human amniotic membrane transplantation: past, present, and future. Ophthalmol Clin North Am. 2003 March; 16(1):43-65, vi.). Dried or freeze dried amnion may be rehydrated prior to use by soaking in sterile saline. Some dried amnion products may not need rehydration prior to use, such as BioCover™ (Snoasis Medical, Denver, Colo.), which is composed of multiple layers of amnion that is dehydrated and used for treatment of gingival recession of the gums in the mouth. See also U.S. Patent Application Publication No. 2004/0048796 to Hariri et al., which is incorporated by reference herein.

In some embodiments, harvested amnion is processed by removing the chorion and chemically and mechanically cleaning the remaining inner amnion collagen sheet (e.g., with normal saline and antibiotics). The inner amniotic membrane consists of a single layer of epithelium cells, thin reticular fibers (basement membrane), a thick compact layer, and a fibroblast layer. The basement membrane contains collagen types III, IV, and V and cell-adhesion bioactive factors including fibronectin and laminins (e.g., laminin-5). In some embodiments, the tissue is processed by removing the epithelium layer of the inner amniotic membrane. See, e.g., WO2009/033160.

In some embodiments, the amnion is processed to be decellularized, which decellularization may be performed by methods known in the art.

Amnion or other collagen components may be provided as multiple layers to provide a moldable consistency. The components may be optionally dried for packaging, to be rehydrated and molded just prior to use.

### B. Collagen Deposition Inhibitor

In some embodiments, the barrier material includes a collagen deposition inhibitor component. “Collagen deposition inhibitors” useful for carrying out the present invention are known and include all agents that inhibit the synthesis of collagen. See, e.g., US Patent Publication No. 2009/0028914; U.S. Pat. Nos. 6,046,340 and 5,092,841; PCT Publication No. WO/2005/112999.

In some embodiments, inhibitors of type-1 collagen (also known as Type I collagen) deposition are preferred. The primary component of scar tissue, collagen type-1 alpha, typically forms a protein rod 300 nm long composed of 3 subunits: two a α1(I) chains and one α2(I) chain. Within the fibroblast, elaboration of type-1 collagen is controlled by activation of the alpha-1 collagen gene. Therefore, in some embodiments, inhibitors of the alpha-1 collagen gene expression are preferred.

Examples of “collagen deposition inhibitors” as used herein include, but are not limited to, mithramycin, mitomycin-c, tranilast, halofuginone, d-penicillamine, beta-aminopropionitrile, okadaic acid, LY294002 (PI-3K inhibitor), 5-fluorouracil, analogs thereof, etc.

Mithramycin (MIT or plicamycin) is an aureolic acid polyketide antibiotic that binds to GC-rich areas of DNA, and is typically used as a chemotherapeutic agent. See, e.g., U.S. Pat. No. 5,723,448.

Mitomycin-c is a known fibroblast inhibitor with known scar inhibitory effects in the eye, sinus and trachea.

Tranilast (2-(2,3-dimethoxycinnamoyl)aminobenzoic acid) is also known and described in, for example, U.S. Pat. Nos. 5,385,935; 6,239,177; and 6,376,543.

Halofuginone, or halofuginone bromide (7-bromo-6-chloro-3-[3-(3-hydroxy-2-piperidinyl)-2-oxopropyl]-4(3H), is known and described in, for example, U.S. Pat. Nos. 5,449,678, 6,420,371; 6,028,078; 6,090,814; and 6,159,488. Halofuginone is a quinazolinone compound that has been used in the cattle and poultry industries as an anti-coccidal agent. Serendipitously, it was discovered that dermal thinning was occurring in chickens that were administered the drug systemically. Further study of this phenomenon led to the discovery that the mechanism of action of halofuginone was inhibition of the alpha-1 collagen gene promoter (Granot I et al. Poult Sci. 1991 July; 70(7):1559-63). The pharmacology of this compound has been extensively studied for veterinary use and has FDA orphan drug approval for use in humans to treat scleroderma. Halofuginone has been reported to prevent adhesions in a rat model in a dose dependent manner when treated with either oral or intraperitoneal injections of halofuginone for approximately one week prior to surgery and for three weeks postoperatively (Nagler et al., Am J Obstetric Gynecol 1999; 180:558-63).

In some embodiments, collagen deposition inhibitors are provided as a coating (i.e., a layer or film on a surface) on a substrate (e.g., the collagen component and/or chitin component). Collagen deposition inhibitors may be coated on a substrate by any suitable technique, such as dipping, spraying, spray drying, etc. The collagen deposition inhibitor may be applied per se or concurrently with a carrier material or gel-forming or film-forming material, such as a biodegradable polymer (e.g., alginate). Collagen deposition inhibitors may be combined into materials (such as powders or biodegradable materials) by any suitable technique, such as mixing, co-extruding, etc.

In preferred embodiments, the collagen deposition inhibitor is included in an amount effective to inhibit or effect scar formation and/or collagen formation locally, i.e., on or adjacent to the implanted or inserted barrier. In some embodiments, compositions including collagen deposition inhibitors may be administered via a coated collagen component as described above, via combination with a gel or suitable wound glue, via coatings and/or impregnating collagen deposition inhibitors onto a suitable substrate or barrier material as described herein.

In some embodiments, the collagen deposition inhibitor is administered or provided in a range from nano (10⁻⁹) to pico (10⁻¹²) molar doses. In some embodiments, local application of one or more collagen deposition inhibitors in the range of nano (10⁻⁹) to pico (10⁻¹²) molar doses is sufficient to inhibit collagen type-1 production locally and thereby prevent, inhibit or treat adhesions. In some embodiments, the collagen deposition inhibitor is 10⁻⁹, 10⁴⁰, 10⁴¹, or 10⁻¹² molar. In some embodiments, the collagen deposition inhibitor is 10⁻⁹ to 10⁴⁰, or 10⁻⁹ to 10⁻¹¹, or 10⁻¹⁰ to 10⁻¹¹, or 10⁻¹⁰ to 10⁻¹², or 10⁻¹¹ to 10⁻¹² molar.

In some embodiments, collagen deposition inhibitors are administered by elution/absorption of the drug in less than about 30 minutes. In some embodiments, administration is performed over a longer period of time, e.g., substantial elution over 30 minutes, 1, 2 or 3 hours, and up to 5, 6, 7 or 8 days. In some embodiments, collagen deposition inhibitors are eluted over time to capture as much of the early fibroplasia stage of wound healing as possible (e.g., over 3-7 days).

### C. Chitosan

In some embodiments, the barrier material includes a component of chitin or derivatives thereof, including, but not limited to, chitosan. “Chitin,” or poly-N-acetylglucosamine, a natural polysaccharide, forms the cell walls of fungi and the hard shell of insects and crustaceans. “Chitosan” is linear derivative of chitin composed of randomly distributed β-(1-4)-linked D-glucosamine (deacetylated unit) and N-acetyl-D-glucosamine (acetylated unit), and can be made, e.g., by the deacetylation of chitin, as known in the art.

The use of chitin and its derivatives for wound healing is known (see, e.g., U.S. Pat. Nos. 3,232,836, 3,632,754, 3,903,268 and 6,150,581). Chitosan is an effective component of a bandage preparation for treating trauma-induced hemorrhage, and does not induce tissue injury or adhere to the underlying tissue, and has been shown to have bacteriostatic properties.

Chitosan has also been shown to effectively reduce adhesion formation in rat models of tissue abrasion, tissue ischemia and tissue infection (Zhou et al. Preventive effect of gelatinizedly-modified chitosan film on peritoneal adhesions of different types. World J Gastroenerol 2007; 13:1262-7). Without wishing to be bound by theory, one possible mechanism of action of chitosan's adhesion prevention is its ability to prevent fibroblast and macrophage adherence to the injury site (see Zhou et al. Reduction in postsurgical adhesion formation after cardiac surgery in a rabbit model using N,O-carboxymethyl chitosan to block cell adherence. J Thorac Cardiovasc Surg 2008 April; 135(4):777-83).

In some embodiments, chitosan may be provided with the collagen deposition inhibitor or other components in situ, e.g., in the form of a spray, optionally including a carrier (e.g., alginate). See U.S. Pat. Nos. 6,150,581, 5,266,326.

### II. Combinations

As noted above, provided herein are multimodal adhesion barriers, inclusive of combinations of at least two of the above-listed components, so combined by mixing, incorporating and/or layering with respect to one another. Combinations of these components may be prepared as noted above or variations thereof that will be apparent to those of skill in the art, and may be optimized for a particular application.

In some embodiments, the multimodal adhesion barrier composition is tailored to the surgical application. For example, in some embodiments, the barrier includes a chitosan component to promote thrombosis and act as an anti-inflammatory for indications in which these functions are deemed needed by the surgeon (e.g., CAB, (C+B)A(C+B), (C+B)A, etc.). Other hemostasis agents may also be included, e.g., thrombin, keratin, fibrin, etc.

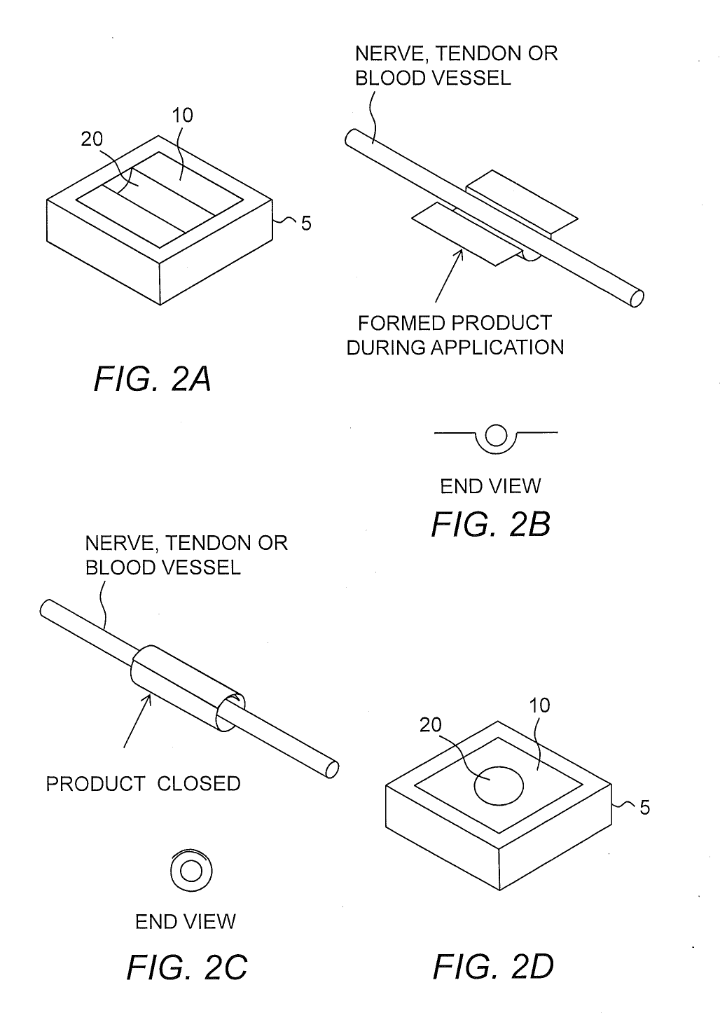

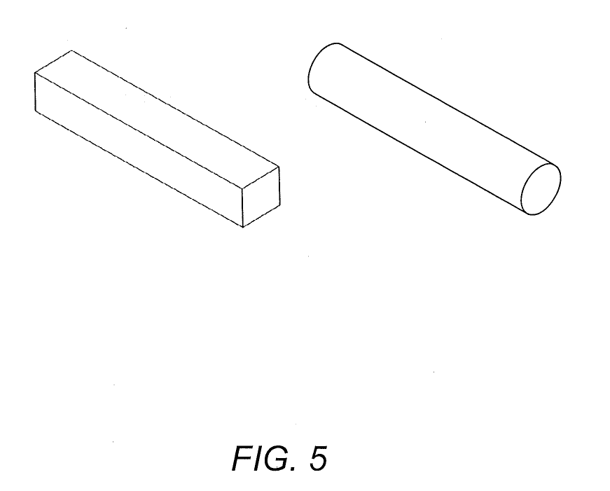

The barriers in some embodiments may be shaped according to intended used and then dried for packaging. Formed barriers (e.g., tubes, wraps, sheaths, etc., optionally dried or dehydrated) may then, in some embodiments, be packaged to create a “preformed” barrier for subsequent use during surgery (e.g., orthopedic, tendon repair, nerve repair surgery, etc.). In some embodiments, an open cylinder (see, e.g., FIG. 2) is provided, optionally shaped prior to use. In some embodiments, a shapeable plug is provided (see, e.g., FIG. 5). An applicator may also be used to aid in holding and placing the barrier during surgery.

In some embodiments, a mold may be provided to shape the barrier prior to application (see, e.g., FIG. 2). The mold (5) may comprise a flat or substantially flat portion (10) adjacent to one or more sides of a concave portion (20) of the mold. The shaped barrier may then be more easily handled and applied to the tissues during surgery, particularly to a nerve, tendon, blood vessel or other tubular structure, and/or to a rounded tissue or area of the body such as an organ.

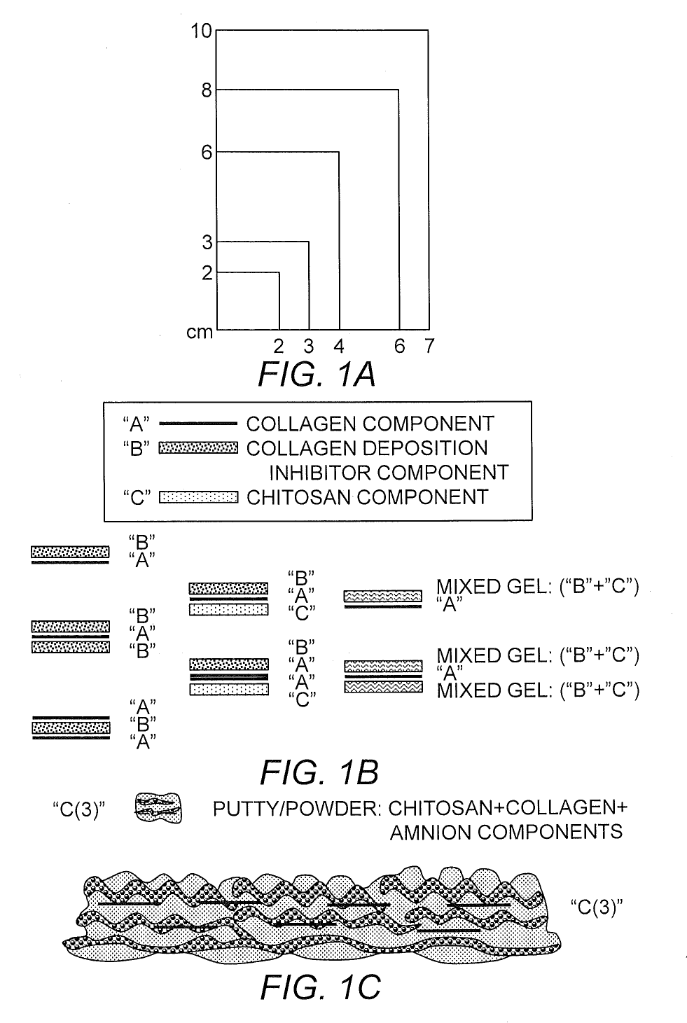

In some embodiments, sheets are provided that can optionally be cut to shape prior to use (see, e.g., FIG. 1A). In some embodiments, a biocompatible glue may be used to aid in attachment of the barrier to the intended tissue. See, e.g., U.S. Pat. No. 6,428,561.

In some embodiments, the barrier has a viscosity or consistency similar to toothpaste or modeling clay. In some embodiments, the viscosity of the composition is fluid and malleable and able to hold a form or shape without a supporting structure (e.g., a shapeable plug such as that shown in FIG. 5).

The composition of the present invention may be provided to the user in a dry form, which can be rehydrated for later use. In some embodiments, the components are provided in a powder form that may optionally be rehydrated prior to use, e.g., to form a gel or shapeable plug (see, e.g., FIG. 1C).

Barrier sheets or sheaths according to some embodiments may be provided with a width of between 0.1, 0.5, 1, 2 or 3 mm and 4, 5, 6, 8, 10, 15 or 20 mm, and/or a height of between 0.1, 0.5, 1, 2 or 3 mm and 4, 5, 6, 8, 10, 15 or 20 mm. In some embodiments, sheets may be provided with a width of between 0.1, 0.5, 1, 2 or 3 cm and 4, 5, 6, 8, 10, 15 or 20 cm, and/or a height of between 0.1, 0.5, 1, 2 or 3 cm and 4, 5, 6, 8, 10, 15 or 20 cm.

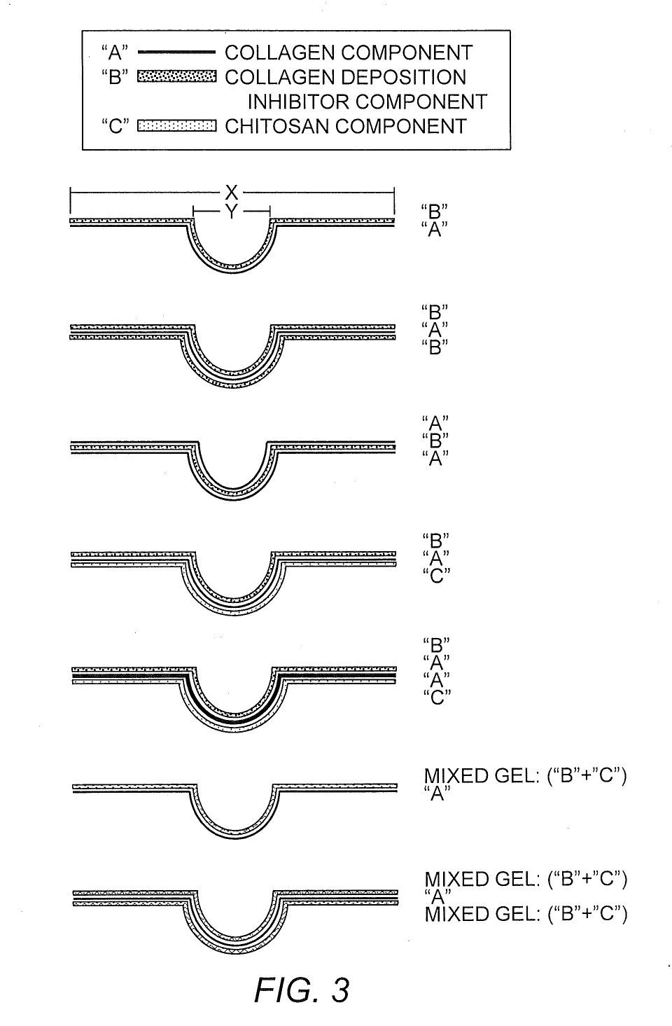

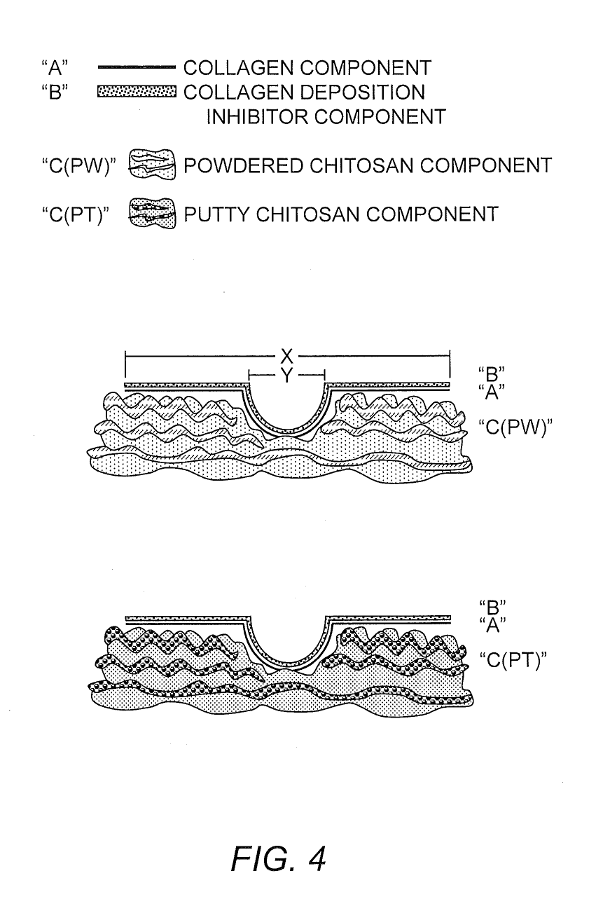

Open cylinders such as the embodiments provided in FIG. 3 may have an overall length of between 0.1, 0.5, 1, 2 or 3 mm and 4, 5, 6, 8, 10, 15 or 20 mm. In some embodiments, the overall length may be between 0.1, 0.5, 1, 2 or 3 cm and 4, 5, 6, 8, 10, 15 or 20 cm. Open cylinders in some embodiments may have a diameter of the semicircular portion of between 0.05, 0.1, 0.5, 1 or 1.5 mm and 2, 2.5, 3, 4, 5, 7.5 or 10 mm, or between 0.05, 0.1, 0.5, 1 or 1.5 cm and 2, 2.5, 3, 4, 5, 7.5 or 10 cm.

The barriers may also be treated with additives or drugs prior to use, for example, to promote the formation of new tissue, lessen bleeding, prevent infection, reduce inflammation, etc. Thus, for example, growth factors, cytokines, antibiotics, thrombolytics, and/or other bioactive materials can be added in or onto the barrier. Such additives will, in general, be selected according to the tissue or organ with which the barrier will likely be in fluid contact.

Some embodiments of present invention are explained in greater detail in the following non-limiting examples.

### EXAMPLES

The effectiveness was tested of amnion coated with halofuginone on both sides or halofuginone on one side and chitosan on the opposite side in preventing peritoneal adhesions and reducing the severity of adhesions that formed in the rat uterine horn injury model.

Collagen substrate and barrier preparation. Patients completed Wake Forest University Health Sciences and Forsyth Medical Center Institutional Review Board approved informed consent during their routine presurgical anesthesia visit for elective cesarean section at term. Following delivery of the infant and placenta, the amniotic membrane was aseptically dissected from each placenta. The chorion was then discarded and the remaining collagen sheet was chemically and mechanically cleaned with normal saline and antibiotics.

The plain amnion sheets (amnion) were constructed by utilizing approximately 200 cm² of the partially decellularized collagen sheet which was then was dried at room temperature in a biological safety cabinet, cut into 2 cm×3 cm rectangles, laid flat in pouches, sealed, labeled, and irradiated.

The halofuginone/amnion/halofuginone sheets (HAH) were constructed by utilizing approximately 200 cm² of collagen sheet and submerging it in the halofuginone gel solution and refrigerated at 4° C. for 10 hours, followed by drying in a biological safety cabinet. The coated matrix was cut into 2 cm×3 cm rectangles, laid flat in pouches, sealed, labeled, and irradiated. The halofuginone coating was made by mixing 1.5 gram of sodium alginate (Spectrum Chemical, Gardena Calif.) into 75 ml of sterile distilled water (Baxter, Deerfield Ill.). The mixture was heated and agitated to form a flowing gel. The gel was cooled to room temperature. 20 ml of gel was mixed with 20 ml halofuginone 0.5 mg/ml solution (Halocur®, Intervet, Intervet Ireland Ltd.) to form a viscous liquid with uniform color.

The chitosan/amnion/halofuginone sheets (CAH) were constructed by utilizing approximately 200 cm² of collagen sheet. The chitosan gel mixture was spread in a uniform manner across a dying fixture. A prepared layer of the collagen sheet was placed on top of that, and then a top layer of halofuginone gel was spread upon it. The resulting configuration was placed in a biological safety cabinet and dried, then cut into uniform rectangles, laid flat in pouches, sealed, labeled, and irradiated. Chitosan gel was made by mixing 0.5 g of Chitosan (Tokyo Chemical Industry Co, Ltd Tokyo, Japan for TCI America) was added to 30 ml Acetic Acid, 1% Aqueous solution (Electron Microscopy Sciences, Hatfield, Pa.), heated and agitated to form a gel, then cooled to room temperature.

The chitosan/amnion/chitosan sheets (CAC) were constructed by utilizing approximately 200 cm² of collagen sheet. The chitosan gel mixture was spread in a uniform manner across a dying fixture. A prepared layer of the collagen sheet was placed on top of that, and then a top layer of chitosan gel was spread upon it. The resulting configuration was placed in a biological safety cabinet and dried at room temperature, then cut into uniform rectangles, laid flat in pouches, sealed, labeled, and irradiated.

Testing. Sixty retired breeder female Sprague Dawley rats (Charles River Labs, Charles River, N.J.) were randomly assigned to one of six treatment groups: A) Untreated Control; B) Carboxymethylcellulose/hyaluronic acid (CMC/HA) barrier control (Seprafilm™, Johnson and Johnson, Cincinnati, Ohio); C) Plain Amnion; D) Halofuginone/Amnion/Halofuginone; E) Chitosan/Amnion/Halofuginone; F) Chitosan/Amnion/Chitosan Halofuginone.

The animals were kept in single cage housing with an ambient room temperature of approximately 72° F. with twelve hour light and dark cycles and ad lib access to pelleted rat food and drip water. After assignment to treatment groups, each animal was anesthetized with 1.5-2% isoflurane in oxygen general anesthesia w/ spontaneous breathing during the procedure. Following successful anesthesia and utilizing clean surgical technique, each rat's abdominal wall was cleaned with isopropyl alcohol and a 3-4 cm midline abdominal incision was performed. The left uterine horn of each uterus was elevated into the incision. A zone of ischemia was created by crushing each left horn with a hemostat for approximately 30 seconds. The longitudinal vascular bundle of the horn was not compromised. After the uterine horn injury procedure was completed, the injured uterine horn was wrapped in the adhesion barrier of the animal's assigned treatment group (the injured uterine horn of the untreated control animals was not wrapped). The animals were sacrificed two weeks postoperatively using pentobarbital and necropsied. The surgeons did not necropsy the animals they had operated on initially. At necropsy, the animals were assessed for presence or absence of adhesions involving the injured uterine horn and the presence of any adhesion barrier material. The adhesions that had formed were assigned a severity score of: 1—filmy, did not require countertraction for lysis; 2—required countertraction for lysis; or 3—required sharp dissection for lysis.

Statistical methods. Prior studies have demonstrated that an n of 10 animals per treatment group is sufficient to determine a significant treatment effect at a level of p<0.05 with 90% power (see Szabo et al. Eur Surg Res 2000; 32:125-8; Nagler et al. Am J Obstetric Gynecol 1999; 180:558-63; Zhou et al. World J Gastroenerol 2007; 13:1262-7) Percent animals in a treatment group with adhesions to the injured uterine horn and percent age of adhesions that were dense (grade 3, requiring counter traction or sharp dissection to lyse) were compared to the untreated and CMC/HA controls by chi square analysis. A p value of less than 0.05 was considered statistically significant.

Results. As depicted in Table 1, both the halofuginone/amnion/halofuginone and the chitosan/amnion/halofuginone barriers prevented adhesions when compared to either untreated or CMC/HA treated controls. Table 2 depicts the percent of the adhesions that formed that were dense adhesions (grade 3). Some animals formed several adhesions to the injured uterine horn.

There were no adhesions formed in the halofuginone/amnion/halofuginone group and no dense adhesions formed in the chitosan/amnion/halofuginone group.

While neither the plain amnion barrier nor the chitosan/amnion/chitosan barrier significantly reduced adhesion formation, both groups had significantly fewer dense adhesions compared to untreated and CMC/HA barrier treated controls.

There was no evidence of any residual adhesion barrier in any animal at necropsy.

TABLE 1

| | Un tx | CMC/HA | | | | |
|---|---|---|---|---|---|---|
| | Control | control | Amnion | HAH | CAH | CAC |
| | n = 9 | n = 7 | n = 9 | n = 9 | n = 7 | n = 10 |
| Percent | 55.56 | 71.43 | 66.67 | 0** | 14.29* | 50 |
| animals | | | | | | |
| w/adhesions | | | | | | |

TABLE 2

| | Un Tx | CMC/HA | | | | |
|---|---|---|---|---|---|---|
| | Control | control | Amnion | HAH | CAH | CAC |
| | n = 6 | n = 11 | n = 9 | n = 0 | n = 1 | n = 7 |
| Percent | 100 | 81.81 | 33.33** | 0# | 0** | 14.29** |
| dense | | | | | | |
| adhesions | | | | | | |
| (grade 3) | | | | | | |

*is p < 0.05;
**is p < 0.01;
#could not be analyzed since there were no adhesions formed. In table 2, some animals developed multiple adhesions to the uterine horn injury site.

Discussion. The experiments tested whether a collagen substrate derived from human amniotic membrane alone or coated with chitosan gel (poly-N-acetyl glucosamine) or halofuginone gel (a collagen synthase inhibitor) or coated with chitosan gel on one side and halofuginone gel on the other side would effectively prevent adhesion formation in the rat uterine horn injury model. It was demonstrated that the multimodal bioabsorbable adhesion barriers effectively prevented adhesions and reduce the severity of the adhesions that do form. The two barriers that were effective were constructed with a collagen substrate derived from human amniotic membrane that was coated on both sides with halofuginone gel or was coated on one side with halofuginone gel and the other side with chitosan gel. Without wishing to be bound by theory, it is believed that the efficacy of those two barriers is a result of the physical barrier of the collagen substrate coupled with inhibition of collagen synthesis by halofuginone and the possible prevention of fibroblast and macrophage adherence by the chitosan gel.

The CMC/HA control barrier, plain amnion and CAC barriers were no more effective at preventing adhesions than the untreated controls, but each of the barriers utilizing the amnion derived collagen substrate effectively reduced the formation of the densest adhesions. The reduced efficacy of the plain amnion, CMC/HA control and CAC coated amnion barriers in preventing adhesion formation may be the result of not apposing or attaching the injured uterine horn to another injured peritoneal surface. The injured uterine horn was allowed to remain “free floating” in the abdomen. The decreased efficacy of the other treatments may also have been the result of use of “clean” and not “sterile” surgical techniques. The excellent effectiveness of the HAH and CAH treatments at preventing adhesion formation and reducing adhesion severity scores under these conditions is even more remarkable.

These results support the multimodal approach to adhesion prevention described herein.

The foregoing is illustrative of the present invention, and is not to be construed as limiting thereof. The invention is defined by the following claims, with equivalents of the claims to be included therein.

That which is claimed is:

1. A multimodal barrier material for preventing adhesions in a subject, said material comprising:
(a) collagen; and
(b) a collagen deposition inhibitor coated thereon.

2. The barrier material of claim 1, wherein said material is in the form of a bioabsorbable sheet, tube, open cylinder, wrap, sheath, or plug.

3. The barrier material of claim 1, wherein said collagen comprises amnion.

4. The barrier material of claim 1, wherein said collagen deposition inhibitor is coated on one side of said collagen.

5. The barrier material of claim 1, wherein said collagen deposition inhibitor is coated on both sides of said collagen.

6. The barrier material of claim 1, wherein said collagen deposition inhibitor is selected from the group consisting of: mithramycin, mitomycin-c, tranilast, halofuginone, d-penicillamine, beta-aminopropionitrile, okadaic acid, LY294002 (PI-3K inhibitor), 5-fluorouracil, and analogs thereof.

7. The barrier material of claim 1, wherein said collagen deposition inhibitor is halofuginone.

8. The barrier material of claim 1, further comprising a chitin or a derivative thereof on or in said collagen or said collagen deposition inhibitor.

9. The barrier material of claim 1, further comprising chitosan on or in said collagen or said collagen deposition inhibitor.

10. A method of inhibiting adhesions in a subject in need thereof comprising administering the barrier material of claim 1 to said subject in an amount effective to inhibit said adhesions.

11. The method of claim 10, wherein said adhesions are pelvic or abdominal adhesions.

12. The method of claim 10, wherein said adhesions are peritendinous or perineural adhesions.

13. The method of claim 10, wherein said administering comprises contacting said barrier material to a tissue or positioning said adhesion barrier between tissues during surgery.

14. The method of claim 13, wherein said surgery is abdominal surgery, pelvic or gynecological surgery, orthopedic surgery, ocular surgery, neurosurgery, urologic surgery, cardiothoracic surgery, plastic surgery, veterinary surgery, otolaryngology surgery, podiatric surgery, vascular surgery, trauma surgery, or transplant surgery.

15. The method of claim 10, wherein said barrier material is preformed.

16. The method of claim 10, further comprising the step of shaping said barrier material on a mold prior to said administering.

17. A kit comprising:
(a) the barrier material of claim 1;
(b) a container in which said barrier material is packaged in sterile form; and
(c) optionally, a mold for shaping the barrier material.

18. The kit of claim 17, wherein said container comprises a plastic or foil.

19. The kit of claim 17, wherein said container is vacuum-packed.

20. The kit of claim 17, wherein said barrier material is provided in dehydrated form.

21. The kit of claim 17, wherein said barrier material is coated with a single unit dose of said collagen deposition inhibitor.
